(19) United States
(12) Patent Application Publication
(10) Pub. No.: US 2023/0317286 A1
(43) Pub. Date: Oct. 5, 2023

(54) SYSTEM AND METHOD FOR DIAGNOSING AND STAGING NEURODEGENERATIVE DISEASES ON THE BASIS OF THE SURFACE ROUGHNESS OF RETINAL LAYERS

(71) Applicant: Lucía Jáñez García, Pozuelo de Alarcón (ES)

(72) Inventor: Lucía Jáñez García, Pozuelo de Alarcón (ES)

(73) Assignee: Telepresencia S.L., Madrid (ES)

(21) Appl. No.: 18/024,095

(22) PCT Filed: Sep. 3, 2021
(86) PCT No.: PCT/ES2021/070646

§ 371 Date: Mar. 1, 2023

(30) Foreign Application Priority Data
Sep. 4, 2020 (ES) P202000134

(51) Int. Cl. G16H 50/20; G16H 30/40; G16H 50/70

(52) U.S. Cl. CPC G16H 50/20; G16H 30/40; G16H 50/70

(57) ABSTRACT

A system and method for diagnosing neurodegenerative diseases and determining their progress comprises automating the process with the following steps: obtaining tomographic data of a region of the retina; segmenting distinguishable layers in the retina; generating a numerical model of the surfaces defined on the retina, their integral layers or regions of same; determining the thickness of the layers and generating a numerical model of the corresponding surface; spatially normalising the obtained surfaces; calculating the roughness of the surfaces using their fractal dimension or an alternative roughness index; and using statistical techniques and algorithms generated by means of automatic learning to diagnose the neurodegenerative disease and determine its progress on the basis of the obtained surfaces and their roughness.

### CROSS-REFERENCE TO RELATED APPLICATIONS

This application is the National Stage of International Application No. PCT/ES2021/070646, filed Sep. 3, 2021, which claims the benefit of Spanish Application No. P202000134, filed Sep. 4, 2020, the contents of which is incorporated by reference herein.

### DESCRIPTION

### Field of the Invention

The invention is of application in medicine and neuroscience and, in particular, for diagnosing and determining the progress of Alzheimer's disease and other neurodegenerative diseases.

### Background of the Invention

The technical problem focuses on the identification of early biomarkers of neurodegenerative diseases such as Alzheimer's disease (AD), amyotrophic lateral sclerosis (ALS), Parkinson's or multiple sclerosis.

The brain is the main tissue affected by neurodegenerative diseases and the retina is the only neuronal tissue that can be analysed non-invasively. There is growing scientific evidence that neurodegenerative pathologies in the retina correspond directly to analogous pathologies in the brain. In patients with mild AD Mutlu et al. (2017) found that thinning of ganglion cell layers (GCL), nerve fibres (NFL) and internal plexiform (IPL) are associated in the brain with a lower volume of grey matter, white matter and hippocampus. Ong et al. (2015) found that a lower total retinal thickness is associated with a lower volume of grey matter only in the temporal lobe of the brain, whilst thinning of the GCL and IPL layers taken together is associated with a lower volume of grey and white matter in the temporal lobe and of grey matter of the occipital lobe. Casaletto et al. (2017) found that thinning of the retina and its GCL layer is related to medial temporal lobe atrophy. In a homogeneous sample of patients with early stage AD, Salobrar-Garcia et al. (2015) found that the development of AD leads to a reduction in the total thickness of the retina in the peripapillary region. Garcia-Martin et al. (2014) have found that there already appears a thinning of the retina in the macular area at a very early stage of AD, together with a 40% decrease in contrast sensitivity (Salobrar-Garcia et al. (2015). All these findings converge to demonstrate that the volume of brain structures involved in AD is related to retinal thickness and visual function and further suggests that damage and neural deposits associated with AD also occur in the retina.

All these findings converge to demonstrate that the volume of brain structures involved in AD is related to retinal thickness and visual function. This suggests that the damage and neural deposits associated with AD may appear in the retina before it does in the brain, implying that the retinal analyses could allow the detection of AD during the asymptomatic preclinical period (Shariflou et al. 2017).

Furthermore, the fact that the Optical Coherence Tomography used in retinal scanning has a spatial resolution of several microns, whilst that of brain scanning techniques such as magnetic resonance or positron emission tomography are in the range of millimetres, allows inferring that the alterations produced by AD may be quantified in the retina before, and with greater precision, than in the brain.

Jáñez et al. (2019) provide a precise delimitation of the regions where the total retina and each of its layers show a statistically significant thinning in patients with AD; they have also provided evidence that almost all retinal layers show thickened regions; with new analytical methods they have shown that the thinned regions are interspersed with those thickened in all layers, except in the inner and outer nuclear segments; and that when comparing the distribution of the thickened and thinned areas of one layer with those of others, a statistically significant tendency of the thinned regions to overlap and those thickened to avoid overlaps appears.

The above findings have been corroborated by Song et al. (2020) having developed a new device whereby they have obtained evidence that AD increases heterogeneity in the internal structure of the retinal nerve fibre layer.

With respect to ALS, Rojas et al. (2019), based also on OCT retinal scanning, found that (1) when comparing the baseline of the ALS group with that of the control group, the thickness of the temporal and inferior macular areas of the inner macular ring appears to be significantly increased; (2) in ALS follow-up versus the ALS baseline, significant macular thinning appears in the inferior areas of the inner and outer macular ring; (3) in ALS follow-up relative to the initial value of ALS, there is significant thinning of the peripapillary retinal nerve fibre layer in the upper and lower quadrants; and (4) ALS patients showed a moderate correlation between some OCT parameters in the peripapillary nerve fibre layer and the revised amyotrophic lateral sclerosis functional rating scale score.

In multiple sclerosis, a reduction in macular volume was found in patients compared to control subjects (Dorr et al., 2011; Gordon-Lipkin et al., 2007). On the other hand, Jimenez et al. (2014) have put the thickness of the retina and its layers in relation to Parkinson's.

The thickening of a retinal layer has been attributed to inflammatory processes and its thinning to the disappearance of neuronal tissue. It has been proposed that neurodegeneration begins by an inflammatory process and ultimately triggers the death and elimination of the affected cells. Under this hypothesis, it turns out that thickened and thinned areas change in size and location as the disease progresses.

In a different field, the quantification of roughness is a highly studied subject in several areas of science and technology: the study of surfaces resulting from the fracture of a rock or the determination of the properties of manufactured metal surfaces are two illustrative examples. As a result of the attention received by the subject, there are innumerable procedures to quantify the roughness of a surface. In our case, and considering the nature and characteristics of the delimiting surfaces of the retina and each of its layers provided by OCT, it has been chosen to use the fractal dimension as a roughness index, without excluding the possibility of using other indices among the many available.

### Explanation of the Invention

In this document, the use of the terms “a”, “an” and “some” and similar references in the context of the description of an embodiment of the invention, and especially in the context of the claims, are to be taken to cover both the singular and the plural, unless otherwise indicated herein or clearly contradicted by context. The terms “includes,” “comprises,” “having,” “including,” “comprising,” “such as” and “with” are to be construed as open-ended terms (i.e. meaning “including, but not limited to”), i.e. they are not to be construed as excluding the possibility that what is described and defined includes more elements, steps, etc. unless indicate otherwise. Mention of ranges and intervals of values herein constitute a shorthand method of referring individually to each value falling within the range, or interval unless otherwise indicated herein, and each separate value is incorporated in the specification as if it were individually cited herein. All methods described in this document may be performed in any suitable order unless otherwise indicated in this document or clearly contradicted by context. The use of examples, or related expressions (e.g. “such as”), solely seeks to better clarify the invention and does not pose a limitation on its scope.

The preferred embodiment included herein represents a known form of carrying out an embodiment of the invention, but there may be modifications thereof that are evident to persons skilled in the art and, hence, the invention may be carried out in another other manner to that specifically described herein. Accordingly, this invention includes all modifications and equivalents of the subject matter in the claims attached hereto as permitted by applicable law. Moreover, any combination of the elements described above in all possible variations thereof is encompassed by the invention unless otherwise indicated herein or clearly contradicted by context.

In order to simplify the presentation in this document we will call “retinal layer” or simply “layer” both the total retina and any of its differentiable layers in the volumetric image; and the term “surface” will be used to refer generically to the external surfaces that delimit the retina or its layers, to any surface defined inside a layer such as the medial surface —which divides it into two layers of identical thickness at each of its points-, or to any region delimited on said surfaces.

With neurodegenerative diseases—such as AD and the others mentioned above —both the total retina and the layers that integrate it show thickening in some regions and thinning in others, which induces roughness on the surfaces that delimit them externally or in those that can be defined inside them, such as the medial surface defined above. In addition, these local thickenings and thinnings change as the disease progresses, which causes the surface roughness to change over time. Such considerations have led to the proposition in this invention that quantification of the surface roughness of the retina and its layers provides an index on which to base the diagnosis of the disease; and the temporal evolution of the value of said index can serve to determine the progress of the disease.

This invention proposes an automated and objective system (FIG. 1) and a method (FIG. 2) to diagnose neurodegenerative disease and determine its progress. The invention is based on quantification of the surface roughness of the retinal layers obtained by any means and in particular from three-dimensional images such as those provided by Optical Coherence Tomography (OCT) or Confocal Microscopy (FCM). The diagnosis of the disease is generated by comparing the retinal roughness obtained at the time of diagnosis with a reference roughness or with that obtained on a previous occasion with the same subject. The progress of the disease is determined by comparing the roughness obtained at the time of the evaluation with a previously determined pattern of progress and reflected in a predictive or classification model.

The method comprises the procedure of segmenting the retina and its layers in the tomography, measuring the thickness of each layer in all the scanned points of the retinal surface, creating the numerical models of its delimiting surfaces and thickness, evaluating the roughness of each surface by calculating its fractal dimension or other indices and finally diagnosing and determining the progress of the disease (which we also call staging). The proposed device implements said method. The proposed system integrates the device into a communications network such as the internet to create new telemedical services provided by the device object of the invention.

### The Method

The described method (FIG. 2) comprises the following steps: obtaining the tomographic data from the computer file (200), segmenting retinal layers (202), obtaining the surfaces of the layers (204), obtaining layer thickness maps (206), spatially normalising (208), evaluating the roughness of the surfaces (210), and finally diagnosing the disease and determining its progress (212).

1. Obtaining the tomographic data file (200). The computer file with the tomographic volume can be obtained directly from the equipment that has previously performed the tomography (108) following the procedure of accessing said data specific to the modality used (OCT, CFM, etc.); it can also be taken from a standardised medical image file (PACS) or from another storage system where it has been previously filed (106).

2. Segmentation of the layers (202). This step of the method includes classifying each voxel of the volume and labelling it with the name of the layer to which it belongs when the voxel is part of the retina or with the label “external” when it is outside it (in the vitreous or in another area without interest).

3. Obtaining the surfaces (204). In this step, a numerical representation is created of the delimiting surfaces of the retinal layers, of the medial surfaces of the retina and of its layers, of any other surfaces defined inside the retina and of its layers, of the surface determined by the thickness of the retina and of its layers, of the surfaces determined by the synthetic images generated from the aforementioned surfaces, and of arbitrarily delimited regions on any of the aforementioned surfaces. As a result, a matrix is obtained whose elements indicate the depth (z-axis coordinate) at which the surface is located at the point of the retina (x- and y-coordinates) corresponding to the row and column in which each element of the matrix is located. The numerical model of a surface can be modified by a frequency or orientation selective spatial filter.

4. Calculation of the thickness of the layers (206). The goal of this stage is to determine the thickness of each layer at each scanned point of the retina. The thickness calculation must include the correction of the spatial distortions introduced by the technique used to generate the volumetric image. As a result, a matrix is obtained for each surface whose elements indicate the thickness of the layer at the corresponding point of the retina.

5. Spatial normalisation (208). Thickness maps and other surfaces defined in the x-y plane for each subject are spatially normalised to ensure that when compared to normative data, previous studies in the same subject, or data from other subjects, the comparison is made between the same anatomical regions.

6. Calculation of roughness (210). The calculation of the roughness of each surface may be carried out by any of the available indices (Zhang et al., 2017), among which the fractal dimension is included; to evaluate this, a wide range of techniques are available, among which box counting is included; in turn, the implementation of this technique has several methods, among which are the IRDBC proposed by Long and Peng (2013) and that of Liu et al. (2014).

The roughness of each surface is quantified omnidirectionally or only in a preferred direction, which may be the direction of one of the axes in the plane on which the surface is defined, the direction of the rapid tomography scan, the direction perpendicular to it in the plane of the retina or another direction chosen arbitrarily at each point of the surface.

Diagnosis and progress of the disease (210). The neurodegenerative disease is diagnosed through a neural network —including perceptron and convolutional network-, which uses the numerical models of the surfaces or a subset thereof as predictor variables.

The neurodegenerative disease is also diagnosed from the surface roughnesses by a classification or regression algorithm generated by automatic learning, including discriminant functions, decision trees, random forests, support vector machines and shallow and deep neural networks. The algorithm establishes the diagnosis of neurodegenerative disease using surface roughness as predictor variables.

The neurodegenerative disease is also diagnosed affirmatively for a subject when the index evaluating the roughness exceeds a certain threshold (close to 2.1 preferably); or when the difference between the obtained value and a reference value, which will have been obtained previously in the same patient or which will have been established as a normal value, becomes statistically significant; or when a scalar function of the roughnesses of the predictor layers takes values in a certain range; said scalar function may consist of the linear combination of the roughness values of the surfaces. Scalar function can be defined based on existing knowledge or can be obtained by supervised automatic learning, using discriminant functions, decision trees, random forests, support vector machines and surface neural networks such as the perceptron or deep ones such as convolutional ones. These algorithms can use both the surfaces defined in the retina or its layers and its roughness values as predictor variables. The range of values for which the AD—or the neurodegenerative disease in question—is diagnosed is that which exceeds a certain threshold whose value may be predetermined; it may also be determined by the value of the scalar function previously obtained in the same subject; or be equal to the minimum value of the scalar function that makes the difference between the current value of the scalar function and another that has been previously obtained in the same patient, or that has been established as a normative reference, statistically significant. The classifier function can also be generated by an automatic learning algorithm.

The progress of the disease is diagnosed by a scalar function or a resulting automatic learning algorithm obtained by procedures analogous to those used for diagnostic functions.

Thus, the progress of the neurodegenerative disease is determined by a neural network —including perceptron and convolutional network—that uses the numerical models of the surfaces or a subset of them as predictor variables and that classifies the corresponding subject in one of the categories or phases contemplated in the evolution of the neurodegenerative disease.

Also, the progress of the neurodegenerative disease is determined by a classification or regression algorithm obtained experimentally by automatic learning, including discriminant functions, decision trees, random forests, support vector machines, and superficial and deep neural networks; the algorithm uses the surface roughnesses as predictor variables and classifies the corresponding subject in one of the categories or stages contemplated in the evolution of the neurodegenerative disease.

Once the automated analyses have been completed, all the results obtained are recorded on permanent support and made accessible to the user in local or remote mode using the most appropriate methods in each case.

Advantages of the method described herein

1) The method proposed here is fully automated, so it eliminates the variability and subjective biases of manual methods.

2) The quantification of the surface roughness of the retina and its layers by the fractal dimension of their surfaces is an index that has not been previously used and is capable of summarising in a single numerical value the affectation suffered by the entire layer as a whole, which gives it direct clinical usefulness and places it ahead of other indices based only on the thinning or thickening observed in small regions of some layers.

3) Diagnostic and disease phase determination algorithms simultaneously take into account the alterations of all layers by weighting them in an efficient manner, unlike methods based on a single or a small number of layers or regions.

4) The performance of the method in the first embodiment of the invention has been very high, having correctly classified all available cases.

5) The quantification of the surface roughness based on OCT allows obtaining information on the loss of structural homogeneity of the retinal layers without the need for new devices complementary to OCT such as the one proposed by Song et al. (2020) to evaluate the internal destructuring produced in a retinal layer by neurodegenerative diseases.

Device

A computer or computing device, or any other form of programmable hardware, that implements the steps of the method described herein.

FIG. 1 provides a schematic of the structure of modules that integrate the device of one embodiment of the invention consisting of a computer (100) having its own computing unit or CPU (122), its working memory (124), its input devices including mouse and keyboard (126), its output devices including displays capable of displaying data and images (128), a non-volatile file system for storing data and results (130), a non-volatile and tangible medium for program filing (110) that can be read and executed by the CPU with its working memory; said non-volatile, tangible and computer-readable medium for program filing (110) contains the program modules wherein the methods of obtaining the tomography (111), segmentation of the retinal layers (112), calculation of the surfaces (113), calculation of the thickness of the layers (114), spatial normalisation (115), calculation of the roughness (116) and diagnosis and staging (117) are materialised; all the modules that integrate the computer are connected to an internal communication bus (132) that puts all of them in bidirectional communication with each other and with the CPU and the working memory; moreover, through the external communication card (134) the computer also communicates with the external communication networks (136), thus being able to obtain directly through the tomographic data files where they are stored (106) and even be managed by users who interact from their own computers through the internet (104) or from a remote workstation (102).

System

A system comprising the device described in the preceding paragraph and a web server, connected by a network to said device and by another to the internet or other telematic networks; said server a) allows remote users to connect to it, receive a request for diagnosis or staging and the tomographic volume to be analysed, together with the complementary information; b) transfers the request and the tomographic volume to the device to perform the analysis of the tomography and produce the diagnosing and staging; c) receives the results in electronic format from the device; d) transfers them to the applicant through the telematic channel or another that it has selected; and e) reports the completion of the process to the information and management systems of the service provider.

### BRIEF DESCRIPTION OF THE DRAWINGS

To complement the present description, and to help to better understand the characteristics of an embodiment of the invention, said description is accompanied, as an integral part, by a set of drawings wherein the following has been represented in an illustrative and non-limiting manner:

FIG. 1.—Shows a diagram of the structure of modules that integrate the device of an embodiment of the invention, in addition to the functionality of each of the modules.

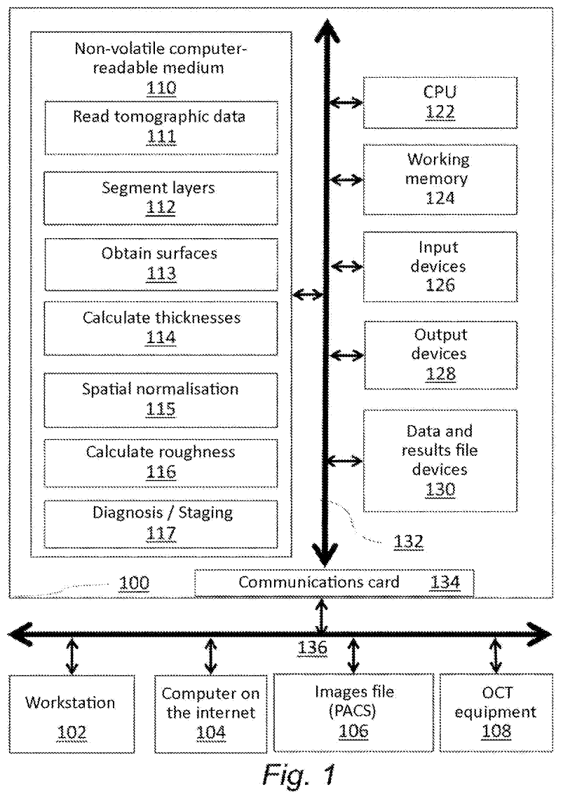

FIG. 2.—Shows the steps that integrate the method in an embodiment of the invention.

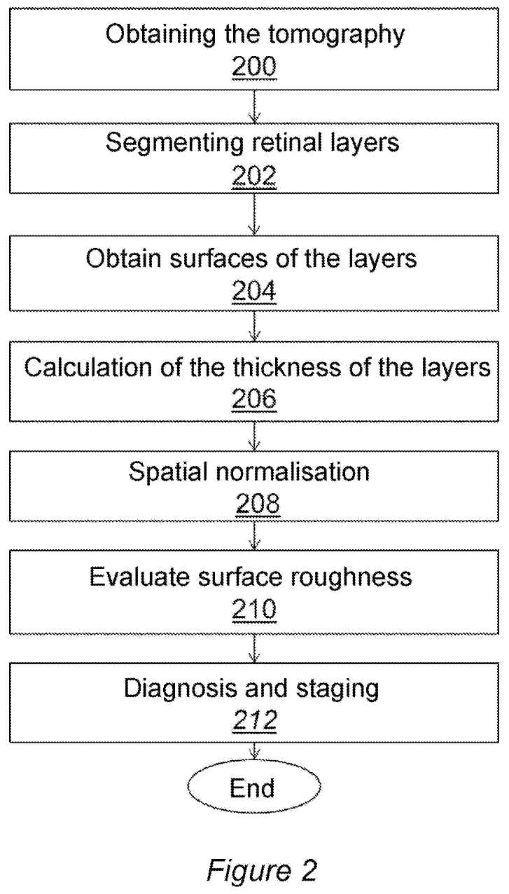

FIG. 3.—Shows the confusion matrix and the performance indices of the diagnostic algorithm used in the preferred embodiment.

### PREFERRED EMBODIMENT OF THE INVENTION

While the invention is described and illustrated in a preferred embodiment, namely in its application to the diagnosis of Alzheimer's based on the fractal dimension of the roughness calculated on the thickness of the retina and 10 segmented layers therein, the invention can be applied and produced with many different configurations. A preferred embodiment of the invention is represented in the drawings, and will be described herein in detail, understanding that the present description is to be considered as an exemplification of the principles of the invention and the associated functional specifications for its construction and it is not to be understood that the invention is limited to the illustrated embodiment but will also encompass equivalent embodiments, even applied in other areas. Persons skilled in the art will envision many other possible variations within the scope of the present invention.

The preferred embodiment described below is an implementation of the method using the roughness of the surfaces defined by the thickness of the retina and its layers.

1. Obtaining the tomographic data file (200). The computer files with the optical coherence volumes with which the preferred embodiment of this invention was constructed were generated with a spectral domain OCT (3D OCT-IOOO Topcon, Japan) performed on 23 normal subjects and 19 subjects affected by AD. Each volume covers a fovea centralis retinal area of 6×6 mm with lateral scanning a scanning density of 512×128 pixels. The voxel size was 11.7×46.9*3.5 μm (horizontal×vertical×depth).

2. Segmentation of the layers (202). The retinal layers were segmented using software called Layer Segmentation Module (Iowa Reference Algorithms 3.6 Retinal Image Analysis Lab, Iowa Institute for Biomedical Imaging, Iowa City, IA, USA). 10 retinal layers were delimited with it: (1) nerve fibres (NFL), (2) ganglion cells (GCL), (3) inner plexiform (IPL), (4) inner nuclear (INL), (5) outer plexiform (OPL), (6) outer nuclear (ONL), (7) inner segments/outer segments (IS/OS), (8) outer segments (OSL), (9) outer segment PR/RPE complex (OPR), (10) retinal pigment epithelium (RPE) layer, and (11) total retina.

3. Obtaining the surfaces (204). The xml files resulting from the software used in the previous stage were decoded with a program written in Matlab to obtain the 3D coordinates of the two delimiting surfaces of each layer, as well as the macular and papillary centres, and the masks for the retinal regions to be excluded in each volume of OCT as the automatic segmentation of the layers was not successful in them.

4. Calculation of the thickness of the layers (206). For each retinal layer, its thickness was measured using the method and software in Matlab described in Jáñez et al. (2019). The thickness of each layer was determined at the same 128×512 points regularly spaced of the scanned retinal area. In this way, each layer was associated with the surface defined by a matrix whose elements indicate the thickness in microns of that layer at the retinal point corresponding to each element of the matrix. The dot layer thickness was corrected to eliminate the excess thickness introduced by the OCT technique as a result of the overall OCT tilt, the natural curvature of the retina, and the variation in the local tilt of its layers. The methodology used for these corrections, as well as the justification for them, is found in the aforementioned publication. The methodology followed is that described in Jáñez et al. (2019) and was performed with its own programs written in Matlab.

5. Spatial normalisation (208). The thickness maps in the x-y plane for each subject were spatially normalised by translation, rotation and dilation to match the foveal centralis of all subjects and to match the angle and length of the maculopapillary axis of all of them. The methodology is as described in Jáñez et al. (2019) and was performed with its own programs written in Matlab.

6. Calculation of roughness (210). The calculation of the roughness of each surface defined by the thickness of each layer was carried out following the “box counting” technique using the Integer Ratio Differential Box Counting (IRDBC) algorithm proposed by Long et al. (2013), taking into account the result of the comparative analysis of this class of algorithms carried out by Panigrahy et al. (2020). The algorithm has been implemented in Matlab.

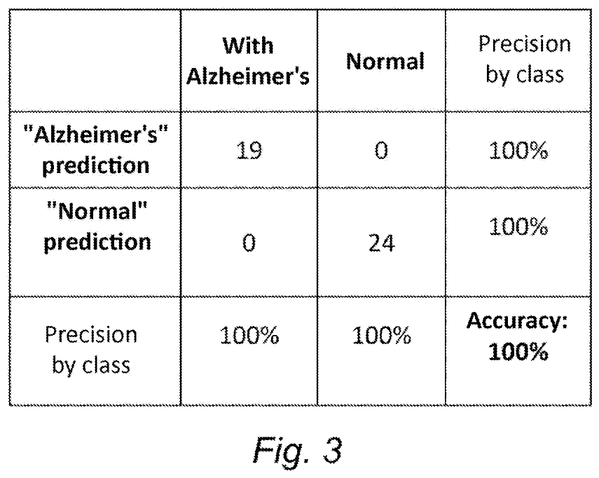

7. Diagnosis and staging (212). The AD was diagnosed using a support vector machine with a radial kernel that was trained with the available cases and that managed to correctly classify all of them (FIG. 3).

How the Invention is Susceptible to Industrial Applicability

The channels currently envisaged for industrial applicability of this invention include: 1) implementation of the method through software programs in diagnostic imaging stations, to aid diagnosis; 2) implementation of the method in tomographic devices, such as OCT equipment; 3) implementation of a web server capable of receiving the tomographic study generated in another remote equipment, perform segmentation and analysis of roughness, generate the diagnosing and staging of the disease, and deliver the results of the study to the study applicant through local or internet telematic networks or any other means and in the necessary format (3D images, reports, etc.); 4) incorporation of the methods of the invention into medical teaching and autonomous learning devices; 5) development of equipment for mass use and automated by the population for early warning of possible neurodegenerative pathologies; 6) marketing and installation of early detection equipment in regions or countries with a shortage of medical specialists; 7) remote diagnostic aid service for the analysis of tomographies generated in equipment lacking the software that implement this method of early diagnosis.

### REFERENCES

Casaletto, K. B. et al. Retinal thinning is uniquely associated with medial temporal lobe atrophy in neurologically normal older adults. Neurobiol. Aging 51, 141-147 (2017).
Derr, J., Wernecke, K. D., Bock, M., Gaede, G., Wuerfel, J. T., Pfueller, C. F., Bellmann-Strobl, J., Freing, A., Brandt, A. U., & Friedemann, P. (2011). Association of retinal and macular damage with brain atrophy in multiple sclerosis. PLoS ONE. https://doi.org/10.r371/joijrrial.pone.0018132.
Esteller, R., Vachtsevanos, G., Echauz, J., & Litt, B. (2001). A Comparison of waveform fractal dimension algorithms. IEEE Transactions on Circuits and Systems I: Fundamental Theory and Applications. https://doi.org/10.1109/81.904882.
Garcia-Martin, E. S. et al. Macular Thickness as a Potential Biomarker of Mild Alzheimer's Disease. Ophthalmology 121, 1149-1151 (2014).
Gordon-Lipkin, E., Chodkowski, B., Reich, D. S., Smith, S. A., Pulicken, M., Balcer, L. J., Frohman, E. M., Cutter, G., & Calabresi, P. A. (2007). Retinal nerve fiber layer is associated with brain atrophy in multiple sclerosis. Neurology.
https://doi.org/10.1212/01.wd 0.0000295995.46586.ae.
Jáñez-Escalada L., et al. “Spatial analysis of thickness changes in ten retinal layers of Alzheimer's disease patients based on optical coherence tomography”. Scientific Reports 9 (2019): 13000. https://doi.org/10.1038/s41598-019-49353-0.
Jiménez, B., Ascaso, F. J., Cristobal, J. A., & Lopez del Val, J. (2014). Development of a prediction formula of Parkinson disease severity by optical coherence tomography. Movement Disorders. https://dpi.prg/10d.Q02/nids.25747.
Li, Y., & Huang, R. (2015). Relationship between joint roughness coefficient and fractal dimensión of rock fracture surfaces. International Journal of Rock Mechanics and Mining Sciences. 0.007.
Liu, Y., Chen, L., Wang, H., Jiang, L., Zhang, Y., Zhao, J., Wang, D., Zhao, Y., & Song, Y. (2014). An improved differential box-counting method to estimate fractal dimensions of gray-level images. Journal of Visual Communication and Image Representation.
https://doi.org/10.1016/j-jvcir.2014.03.008
Long M, Peng F. A box-counting method with adaptable box height for measuring the fractal feature of images. Radioengineering 2013; 22(1):208-13.
Mutlu, U. et al. Retinal neurodegeneration and brain MRI markers: the Rotterdam Study. Neurobiol. Aging. 60, 183-191 (2017).
Ong, Y. T. T. et al. Retinal neurodegeneration on optical coherence tomography and cerebral atrophy. Neurosci. Lett. 584, 12-16 (2015).
Panigrahy C. Seal Ayan, Mahato, N K, Fractal dimension based parameter adaptive dual channel PCNN for multi-focus image fusión, Optics and Lasers in Engineering, Volume 133, 2020, 106141, ISSN 0143-8166, https://doi.org/10.1016/i.optlaseflg.2020.106141.
Rojas P., et al. “Changes in Retinal OCT and Their Correlations with Neurological Disability in Early ALS Patients, a Follow-Up Study”. Brain Sciences 9 (2019): 337.
Salobrar-Garcia, E. et al. Ophthalmologic Psychophysical Tests Support OCT Findings in Mild Alzheimer's Disease. J. Ophthalmol. 2015, Article ID 736949, 10 pages (2015).
Salobrar-Garcia, E. et al. Analysis of Retinal Peripapillary Segmentation in Early Alzheimer's Disease Patients. Biomed Res. Int. 2015, 1-8 (2015).
Shariflou, S. et al. Diagnostic and Prognostic Potential of Retinal Biomarkers in Early On-Set Alzheimer's Disease. Curr. Alzheimer Res. 14, 1000-1007 (2017).
Song G, et al “Multimodal Coherent Imaging of Retinal Biomarkers of Alzheimer's Disease in a Mouse Model,” Scientific Reports, May 13, 2020. DOI: 10.1038/s41598-020-64827-2.

Zhang X, Xu Y, Jackson RL. An analysis of generated fractal and measured rough surfaces in regards to their multi-scale structure and fractal dimensión. Tribol Int 2017; 105:94-101

1. A method for diagnosing and staging neurodegenerative diseases based on the surface roughness of retinal layers comprising:
obtaining a tomographic data file,
segmenting retinal layers in the tomographic volume,
obtaining a numerical representation of the surfaces
calculating a thickness of the layers, determining a thickness map of each layer at each scanned point of the retina and generating a matrix whose elements indicate the thickness of the layer at the corresponding point of the retina,
spatially normalising the obtained surfaces;
calculating a roughness of said surfaces, and
diagnosing the neurodegenerative disease and determining its progress using statistical techniques to compare the values of roughness with normative values or with those from a previous study of the same tissue or using classification or regression models generated by automatic learning, and taking as input variables the numerical models of the surfaces or their roughness indices;
wherein the previous steps are implemented in a computer.

2. The method according to claim 1, wherein the roughness of the retina or its integral layers is quantified on its delimiting surfaces, on the medial surfaces of the retina and its layers, on any other surfaces defined inside the retina and its layers, on the surface determined by the thickness of the retina and its layers, on the surfaces determined by the synthetic images generated from the aforementioned surfaces, and in regions arbitrarily delimited on any of the aforementioned surfaces.

3. The method according to claim 2, wherein the numerical model of the surface is modified by a frequency or orientation selective spatial filter.

4. The method according to claim 1, wherein the roughness of each surface is quantified omnidirectionally or only in preferred directions.

5. The method according to claim 4, wherein the preferred directions of quantification are selected from:
the direction of one of the axes in the plane whereon the surface is defined,
the direction of the rapid tomography scan,
the direction perpendicular to it in the plane of the retina, or
another direction chosen arbitrarily at each point on the surface.

6. The method according to claim 1, wherein the roughness of each surface is quantified by calculating its fractal dimension in the selected direction.

7. The method according to claim 1, wherein the neurodegenerative disease is diagnosed by a neural network and a convolutional network that uses the numerical models of the surfaces or a subset thereof as predictor variables.

8. The method according to claim 1, wherein the neurodegenerative disease is diagnosed when a scalar function of the surface roughness vector takes values in a certain range.

9. The method according to claim 8, wherein the scalar function is a linear combination of the surface roughness values.

10. The method according to claim 8, wherein the range of values of the scalar function for which the neurodegenerative disease is diagnosed is that which exceeds a certain preset threshold or is determined by the value of the scalar function obtained in a previous evaluation of the same subject.

11. The method according to claim 10, wherein the preset threshold for surface roughness defined by the thickness of a retinal layer is a constant value equal to 2.1.

12. The method according to claim 10, wherein the threshold is the minimum value of the scalar function that makes statistically significant the difference between said value and another previously obtained in the same patient or that has been established as a normative reference.

13. The method according to claim 1, wherein the diagnosis of neurodegenerative disease is made from surface roughnesses by a classification or regression algorithm generated by automatic learning, including discriminant functions, decision trees, random forests, support vector machines, and shallow and deep neural networks, wherein the algorithm establishes the diagnosis of neurodegenerative disease using surface roughness as predictor variables.

14. The method according to claim 13, wherein the classification algorithm is a support vector machine with the kernel maximising its performance, such as radial, Gaussian or polynomial kernel.

15. The method according to claim 1, wherein the diagnosis of the neurodegenerative disease is determined by a neural network that uses the numerical models of the surfaces or a subset of them as predictor variables and that classifies the corresponding subject in one of the categories or phases contemplated in the evolution of the neurodegenerative disease.

16. The method according to claim 1, wherein the progress of the neurodegenerative disease is determined by a classification or regression algorithm obtained experimentally by automatic learning, including discriminant functions, decision trees, random forests, support vector machines, and superficial and deep neural networks; the algorithm uses the roughnesses of the surfaces as predictor variables and classifies the corresponding subject in one of the categories or stages contemplated in the evolution of the neurodegenerative disease.

17. The method according to claim 16, wherein the classification algorithm is a support vector machine with the kernel maximising its performance, such as the radial or Gaussian kernel.

18. System A system for diagnosing and staging neurodegenerative diseases based on the surface roughness of the retinal layers, comprising a computer that implements a method for diagnosing and staging neurodegenerative diseases based on the surface roughness of the retinal layers and a web server, connected by a network to said computer and, in addition, to the internet or other telematic networks; wherein said server has the hardware and server and communication programs that allow it to serve web pages, accept, by internet, connections from remote users, receive the request for analysis, diagnosis and staging from the remote user, and the tomographic volume sent by the remote user containing the tomography or models of the surfaces;
to transfer the request and the tomographic volume to said computer for diagnosis and staging;
to receive the results in electronic format from the device;
to transfer them to the applicant through the telematic channel or another that the latter has selected; and
to inform other computer systems of the completion of the data process associated with it.
